(19) United States
(12) Patent Application Publication
(10) Pub. No.: US 2018/0186752 A1
(43) Pub. Date: Jul. 5, 2018

(54) PROCESSES FOR THE PREPARATION OF PESTICIDAL COMPOUNDS

(71) Applicant: Dow AgroSciences LLC, Indianapolis, IN (US)

(72) Inventors: Qiang Yang, Indianapolis, IN (US); Beth Lorsbach, Indianapolis, IN (US); Yu Zhang, Indianapolis, IN (US); Martin J. Walsh, Indianapolis, IN (US); Jeremy Kister, Indianapolis, IN (US)

(21) Appl. No.: 15/853,066

(22) Filed: Dec. 22, 2017

Related U.S. Application Data
(60) Provisional application No. 62/440,237, filed on Dec. 29, 2016.

(51) Int. Cl. C07D 231/40; C07D 401/04

(52) U.S. Cl. CPC C07D 231/40; C07D 401/04

(57) ABSTRACT

This application relates to efficient and economical synthetic chemical processes for the preparation of pesticidal thioethers. Further, the present application relates to certain novel compounds useful in the preparation of pesticidal thioethers.

### CROSS-REFERENCE TO RELATED APPLICATIONS

This application claims priority under 35 U.S.C. § 119(e) to U.S. Provisional Application Ser. No. 62/440,237 filed Dec. 29, 2016, which is incorporated herein by this reference in its entirety.

### TECHNICAL FIELD

This application relates to efficient and economical synthetic chemical processes for the preparation of pesticidal thioethers. Further, the present application relates to certain novel compounds useful in the preparation of pesticidal thioethers.

### BACKGROUND OF THE DISCLOSURE

There are more than ten thousand species of pests that cause losses in agriculture. The world-wide agricultural losses amount to billions of U.S. dollars each year. Stored food pests eat and adulterate stored food. The world-wide stored food losses amount to billions of U.S. dollars each year, but more importantly, deprive people of needed food. Certain pests have developed resistance to pesticides in current use. Hundreds of pest species are resistant to one or more pesticides. The development of resistance to some of the older pesticides, such as DDT, the carbamates, and the organophosphates, is well known. But resistance has even developed to some of the newer pesticides. As a result, there is an acute need for new pesticides that has led to the development of new pesticides. Specifically, US 20130288893(A1) describes, inter alia, certain pesticidal thioethers and their use as pesticides. Such compounds are finding use in agriculture for the control of pests.

Because there is a need for very large quantities of pesticides, specifically pesticidal thioethers, it would be advantageous to produce pesticidal thioethers efficiently and in high yield from commercially available starting materials to provide the market with more economical sources of much needed pesticides.

### Definitions

As used herein, the term “alkyl” includes a chain of carbon atoms, which is optionally branched including but not limited to C₁-C₆, C₁-C₄, and C₁-C₃. Illustrative alkyl groups include, but are not limited to, methyl, ethyl, n-propyl, isopropyl, n-butyl, isobutyl, sec-butyl, tert-butyl, pentyl, 2-pentyl, 3-pentyl, and the like. Alkyl may be substituted or unsubstituted. It will be understood that “alkyl” may be combined with other groups, such as those provided above, to form a functionalized alkyl. By way of example, the combination of an “alkyl” group, as described herein, with a “cycloalkyl” group may be referred to as an “alkyl-cycloalkyl” group.

As used herein, the term “cycloalkyl” refers to an all-carbon cyclic ring, optionally containing one or more double bonds but the cycloalkyl does not contain a completely conjugated pi-electron system. It will be understood that in certain embodiments, cycloalkyl may be advantageously of limited size, such as C₃-C₆. Cycloalkyl may be unsubstituted or substituted. Examples of cycloalkyl include cyclopropyl, cyclobutyl, and cyclohexyl.

As used herein, the term “aryl” refers to an all-carbon cyclic ring containing a completely conjugated pi-electron system. It will be understood that in certain embodiments, aryl may be advantageously of limited size, such as C₆-C₁₀. Aryl may be unsubstituted or substituted. Examples of aryl include phenyl and naphthyl.

As used herein, “halo” or “halogen” or “halide” may be used interchangeably and refers to fluorine (F), chlorine (Cl), bromine (Br) or iodine (I).

As used herein, “trihalomethyl” refers to a methyl group having three halo substituents, such as a trifluoromethyl group.

### DETAILED DESCRIPTION OF THE DISCLOSURE

The compounds and process of the present application are described in detail below. The processes of the present disclosure can be described according to Scheme 1.

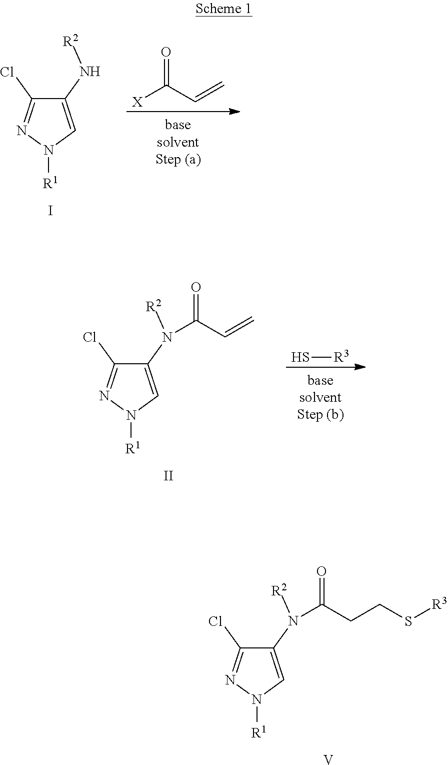

In Step (a) of Scheme 1, the compound of the formula I is acylated with an acryloyl reagent of the formula X—C(O)CH═CH₂, wherein X is a leaving group, such as a halide, —OC(O)C₁-C₆ alkyl, —OC(O)C₆-C₁₀ aryl, and the like, in the presence of a base. The base in Step (a) can be an inorganic base, such as sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), potassium phosphate (K₃PO₄), and the like. Alternatively, the base in Step (a) can be an organic base, such as triethylamine (TEA), diisopropylethylamine (DIPEA), pyridine, and the like. In some embodiments, it can be advantageous to use the base in excess compared to the compound of the formula I. In some embodiments, the base is used in about a 5% molar excess to about a 5-fold excess. In some embodiments, the base is used in about a 3-fold excess. In some embodiments, the base is NaHCO₃. In some embodiments, X in the acryloyl reagent is chlorine. In some embodiments, it can be advantageous to use the acryloyl reagent in excess compared to the compound of the formula I. In some embodiments, the acryloyl reagent is used in about a 5% molar excess to about a 50% molar excess. In some embodiments, the acryloyl reagent is used in about a 10% molar excess to about a 30% molar excess. In some embodiments, the acryloyl reagent is used in about a 20% molar excess.

The reaction of Step (a) can be carried out in the presence of a solvent, or a solvent mixture. Exemplary solvents include, but are not limited to, methylene dichloride (DCM), N,N-dimethylformamide (DMF), tetrahydrofuran (THF), ethyl acetate (EtOAc), acetone, acetonitrile (CH₃CN), dimethylsulfoxide (DMSO), and the like. In some embodiments, the solvent is EtOAc, DCM or THF. In some embodiments, the solvent can be mixed with water. In some embodiments, the solvent is a mixture of THF and water. It can be advantageous to cool the reaction before or during the addition of acryloyl reagent to the reaction mixture. In some embodiments, the reaction is carried out at a temperature of between about −10° C. to about 20° C. In some embodiments, the reaction is carried out at a temperature of between about −10° to about 0° C.

In Step (b) of Scheme 1, the compound of the formula II is reacted with a thiol reagent of the formula HS—R³, wherein R³ is substituted or unsubstituted C₁-C₆ alkyl or substituted or unsubstituted C₁-C₃ alkyl-C₃-C₆ cycloalkyl, in a conjugate addition reaction in the presence of a base. It will be appreciated that C₁-C₆ alkyl and C₁-C₃ alkyl-C₃-C₆ cycloalkyl can be substituted with a wide range of substituents, preferably one or more halogen atoms, preferably one or more fluorine atoms. The base in Step (b) can be an inorganic base, such as sodium bicarbonate (NaHCO₃), sodium carbonate (NaHCO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), potassium phosphate (K₃PO₄), and the like. Alternatively, the base in Step (a) can be an organic base, such as triethylamine (TEA), diisopropylethylamine (DIPEA), pyridine, and the like. In some embodiments, it can be advantageous to use the base in excess compared to the compound of the formula II. In some embodiments, the base is used in about a 5% molar excess to about a 5-fold excess. In some embodiments, the base is used in about a 3-fold excess. In some embodiments, the inorganic base is K₂CO₃.

In some embodiments of Step (b), the thiol reagent is a substituted C₁-C₆ alkyl. In some embodiments, the thiol reagent is a C₁-C₆ alkyl substituted with from 1 to 3 fluorine atoms. In some embodiments, the thiol reagent is 3,3,3-trifluoropropane-1-thiol. In some embodiments, it can be advantageous to use the thiol reagent in excess compared to the compound of the formula II. In some embodiments, the thiol reagent is used in about a 5% molar excess to about a 50% molar excess. In some embodiments, the thiol reagent is used in about a 10% molar excess to about a 30% molar excess. In some embodiments, the thiol reagent is used in about a 20% molar excess. The reaction can be carried out in the presence of a solvent, such as a polar aprotic solvent or a water miscible solvent. Exemplary solvents include, but are not limited to, methylene dichloride (DCM), N,N-dimethylformamide (DMF), tetrahydrofuran (THF), ethyl acetate (EtOAc), acetone, acetonitrile (CH₃CN), dioxane, dimethylsulfoxide (DMSO), and the like. In some embodiments, the solvent is a mixture of water and a water miscible solvent. In some embodiments, the solvent is a mixture of water and dioxane. It can be advantageous to warm the reaction mixture. In some embodiments, the reaction is carried out at a temperature of between about 25° C. to about 75° C. In some embodiments, the reaction is carried out at a temperature of between about 30° C. to about 60° C. In some embodiments, the reaction is carried out a temperature of between about 40° C. to about 60° C.

In some embodiments, R¹ is H. In some embodiments, R¹ is pyridine-3-yl. In some embodiments, R² is H. In some embodiments, R² is ethyl. In some embodiments, R³ is 3,3,3-trifluoropropyl. In some embodiments, R¹ is H and R² is H. In some embodiments, R¹ is pyridine-3-yl and R² is H. In some embodiments, R¹ is H and R² is ethyl. In some embodiments, R¹ is pyridine-3-yl and R² is ethyl. In some embodiments, R¹ is H, R² is H and R³ is 3,3,3-trifluoropropyl. In some embodiments, R¹ is pyridine-3-yl, R² is H and R³ is 3,3,3-trifluoropropyl. In some embodiments, R¹ is H, R² is ethyl and R³ is 3,3,3-trifluoropropyl. In some embodiments, R¹ is pyridine-3-yl, R² is ethyl and R³ is 3,3,3-trifluoropropyl.

### EXAMPLES

### Materials and Methods

These examples are for illustration purposes and are not to be construed as limiting this disclosure to only the embodiments disclosed in these examples.

Starting materials, reagents, and solvents that were obtained from commercial sources were used without further purification. Melting points are uncorrected. Examples using “room temperature” were conducted in climate controlled laboratories with temperatures ranging from about 20° C. to about 24° C. Molecules are given their known names, named according to naming programs within Accelrys Draw, ChemDraw, or

ACD Name Pro. If such programs are unable to name a molecule, such molecule is named using conventional naming rules. ¹H NMR spectral data are in ppm (δ) and were recorded at 300, 400, 500, or 600 MHz; ¹³C NMR spectral data are in ppm (δ) and were recorded at 75, 100, or 150 MHz, and ¹⁹F NMR spectral data are in ppm (δ) and were recorded at 376 MHz, unless otherwise stated.

3-Chloro-1H-pyrazol-4-amine hydrochloride, compound Ia, was prepared according to the method described in U.S. Pat. No. 9,102,655, incorporated herein by reference for the preparation of compound Ia, referred to therein as compound la. 3-Chloro-N-ethyl-1H-pyrazol-4-amine, compound Ib, was prepared was prepared according to the method described in U.S. Pat. No. 9,029,554, incorporated herein by reference for the preparation of compound Ib, referred to therein as compound 7a. 3-(3-Chloro-4-amino-1H-pyrazol-1-yl)pyridine, compound Ic was prepared was prepared according to the method described in U.S. Pat. No. 9,414,594, incorporated herein by reference for the preparation of compound Ic, referred to therein as compound 5d. 3-Chloro-N-ethyl-1-(pyridin-3-yl)-1H-pyrazol-amine, compound Id was prepared was prepared according to the method described in U.S. Pat. No. 9,102,655, incorporated herein by reference for the preparation of compound Id, referred to therein as compound ld.

### CHEMISTRY EXAMPLES

### Example 1

### Preparation of N-(3-chloro-1H-pyrazol-4-yl)acrylamide (IIa)

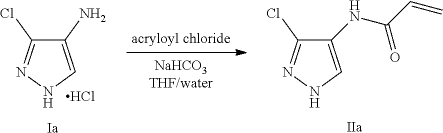

A 4-neck, 500-mL round bottom flask was charged with 3-chloro-1H-pyrazol-4-amine.HCl (15 g, 128 mmol), THF (50 mL), and water (50 mL). Sodium bicarbonate (32.2 g, 383 mmol) was added in portions to control off-gassing, and the mixture was cooled to 5° C. Acryloyl chloride (12.44 mL, 153 mmol) was added at <20° C. and the reaction was stirred for 2 h, after which the reaction was diluted with water (100 mL) and EtOAc (100 mL). The organic layer was concentrated to dryness to afford a white solid, which was suspended in MTBE (50 mL) and stirred for 2 h. The suspension was filtered and the solid was rinsed with MTBE (50 mL) to afford the desired product, N-(3-chloro-1H-pyrazol-4-yl)acrylamide (IIa), as a white solid after drying (14.8 g, 68% yield), mp: 182° C. (decomposition). ¹H NMR (400 MHz, DMSO-d₆) δ 12.96 (s, 1H), 9.77 (s, 1H), 8.10 (s. 1H), 6.58 (dd, J=17.0, 10.2 Hz, 1H), 6.23 (dd, J=17.0, 2.1 Hz, 1H), 5.73 (dd, 10.2, 2.1 Hz, 1H). ¹³C NMR (101 MHz, DMSO-d₆) δ 162.69, 130.76, 130.14, 126.62, 123.60, 116.53. ESIMS: m/z 172.0 ([M+H]⁺).

### Example 2

### Preparation of N-(3-chloro-1H-pyrazol-4-yl)-3-((3,3,3-trifluoropropyl)thio)propionamide (Va)

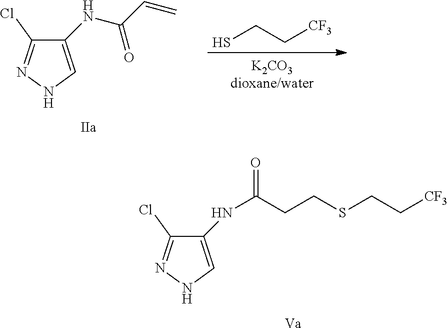 [SMILES: C=CC(=O)Nc1c[nH]nc1Cl ; FC(F)(F)CCS ; "IIa" ; O=C(CCSCCC(F)(F)F)Nc1c[nH]nc1Cl ; "Va"]

To a round bottom flask was added K₂CO₃ (1.77 g, 12.8 mmol), water (4 mL), and dioxane (4 mL). 3,3,3-Trifluoropropane-1-thiol (1.0 g, 7.68 mmol) was added, and the mixture was stirred for 5 min. The above prepared mixture was then added to a 50-mL round bottom flask containing N-(3-chloro-1H-pyrazol-4-yl)acrylamide (1.1 g, 6.41 mmol), dioxane (8 mL), and water (8 mL). The reaction mixture was stirred at 50° C. for 2 h, at which point HPLC analysis indicated that the reaction was complete. The solution was cooled to room temperature and poured into a separatory funnel containing EtOAc (50 mL) and NaHCO₃ (10 mL). The organic layer was separated, and the aqueous phase was extracted with EtOAc (50 mL). The combined organics were washed with brine (25 mL), dried over anhydrous Na₂SO₄ and concentrated under reduced pressure to afford a white solid, which was suspended in MTBE/hexane (1:9, 50 mL) and stirred for 1 h. The solid was collected by filtration, rinsed with hexane (10 mL) to afford the desired product, N-(3-chloro-1H-pyrazol-4-yl)-3-((3,3,3-trifluoropropyl)thio)propionamide (Va), as a white solid (1.79 g, 98% purity, 93% yield). ¹H NMR (400 MHz, DMSO-d₆): 12.89 (s, 1H), 9.58 (s, 1H), 8.00 (s, 1H), 2.81 (t, J=7.0 Hz, 2H), 2.75-2.68 (m, 2H), 2.64 (t, J=7.2 Hz, 2H), 2.61-2.52 (m, 2H). ¹³C NMR (101 MHz, DMSO-d₆): 168.9, 129.9, 126.6 (q, J=277.4 Hz), 123.4, 116.6, 35.2, 33.5 (q, J=27.3 Hz), 26.8, 23.0 (q, J=3.4 Hz). ESIMS m/z 301.8 ([M+H]⁺).

### Example 3

### Preparation of N-(3-chloro-1H-pyrazol-4-yl)-N-ethylacrylamide (IIb)

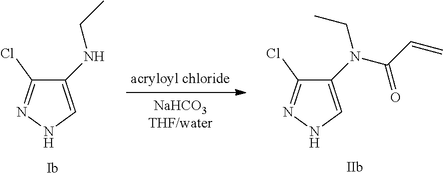

A 4-neck, 100-mL round bottom flask was charged with 3-chloro-N-ethyl-1H-pyrazol-4-amine (2.5 g, 17.17 mmol), THF (10 mL), and water (10 mL). Sodium bicarbonate (3.46 g, 41 2 mmol) was added in portions, and the mixture was cooled to 5° C. Acryloyl chloride (1.34 mL, 16.48 mmol) was added at <20° C. and the reaction was stirred for 2 h, after which it was diluted with water (20 mL) and EtOAc (20 mL). The organic layer was concentrated to dryness to afford a white solid, which was suspended in MTBE (20 mL) and stirred for 2 h. It was filtered and the solid was rinsed with MTBE (10 mL) to afford the desired product N-(3-chloro-1H-pyrazol-4-yl)-N-ethylacrylamide (IIb) as a white solid after drying (2.4 g, 70% yield), mp: 156-160° C. ¹H NMR (400 MHz, DMSO-d₆) δ 8.05 (s, 1H), 6.17 (dd, J=16.8, 2.6 Hz, 1H), 6.06 (dd, J=16.8, 10.0 Hz, 1H), 5.60 (dd, J=10.0, 2.6 Hz, 1H), 3.58 (q, J=7.1 Hz, 2H), 1.03 (t, J=7.2 Hz, 3H). ¹³C NMR (101 MHz, DMSO-d₆) δ 164.82, 136.17, 129.40, 128.02, 127.75, 119.27, 43.29, 12.65. ESIMS: m/z 200.0 ([M+H]⁺).

### Example 4

### Preparation of N-(3-chloro-1H-pyrazol-4-yl)-N-ethyl3-((3,3,3-trifluoropropyl)-thio)propanamide (Vb)

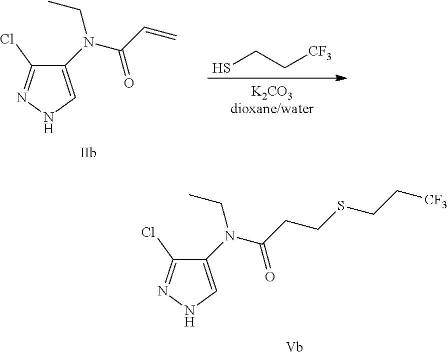 [SMILES: C=CC(=O)N(CC)c1c[nH]nc1Cl ; FC(F)(F)CCS ; "IIb" ; CCN(C(=O)CCSCCC(F)(F)F)c1c[nH]nc1Cl ; "Vb"]

To a round bottom flask was added K₂CO₃ (1.53 g, 11 0 mmol), water (3 mL) and dioxane (3 mL). 3,3,3-Trifluoropropane-1-thiol (0.87 g, 6.69 mmol) was added and the mixture was stirred for 5 min This mixture was added to a round bottom flask containing N-(3-chloro-1H-pyrazol-4-yl)-N-ethylacrylamide (1.11 g, 5.56 mmol), dioxane (7 mL), and water (7 mL). The reaction was stirred for 1 h at 50° C., at which point HPLC analysis indicated complete conversion. The solution was cooled to room temperature and poured into a separatory funnel containing EtOAc (50 mL) and saturated NaHCO₃ solution (10 mL). The organic layer was separated, and the aqueous phase was extracted with EtOAc (50 mL). The combined organics were washed with brine (25 mL), dried over anhydrous Na₂SO₄ and concentrated under reduced pressure to afford a colorless oil. The crude oil was purified by column chromatography (0-80% EtOAc/hexane, R_{f}=0.5 in 6:4 EtOAc/hexane). The fractions containing pure product were concentrated under reduced pressure and co-evaporated with CH₂Cl₂ to afford the desired product N-(3-chloro-1H-pyrazol-4-yl)-N-ethyl3-((3,3,3-trifluoropropyl)-thio)propanamide (Vb) as a colorless oil (1.71 g, 94% purity, 93% yield). ¹H NMR (400 MHz, DMSO-d₆): 13.32 (s, 1H), 8.04 (s, 1H), 3.51 (m, 2H), 2.69 (t, J=7.0 Hz, 2H), 2.63-2.53 (m, 2H), 2.46-2.40 (m, 2H), 2.26 (t, J=7.0 Hz, 2H), 0.99 (t, J=7.1 Hz, 3H). ESIMS m/z 329.9 ([M+H]⁺).

### Example 5

### Preparation of N-(3-chloro-1-(pyridin-3-yl)-1H-pyrazol-4-yl)acrylamide (IIc)

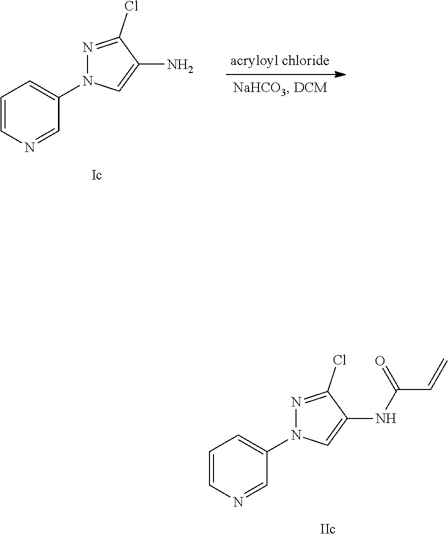 [SMILES: Nc1cn(-c2cccnc2)nc1Cl ; C=CC(=O)Nc1cn(-c2cccnc2)nc1Cl]

A 4-neck, 500-mL round bottom flask was charged with 3-chloro-1-(pyridin-3-yl)-1H-pyrazol-4-amine (14.0 g, 71.9 mmol), and DCM (200 mL). Sodium bicarbonate (18.13 g, 216 mmol) was added, and the suspension was cooled to 0° C. Acryloyl chloride (7.01 mL, 86 mmol) was added at <20° C. and the reaction was stirred for 2 h, at which point HPLC analysis indicated that the reaction was complete. The reaction was quenched with water (100 mL). The suspension was filtered and the filter cake was rinsed with water (2×50 mL). The filter cake was suspended in IPA (200 mL) and stirred at 20° C. for 1 h. Water (200 mL) was added and the resulting suspension was stirred for 2 h. The suspension was filtered and the solid was rinsed with water (2×50 mL) to afford the desired product N-(3-chloro-1-(pyridin-3-yl)-1H-pyrazol-4-yl)acrylamide (IIc) as a white solid after drying (16.8 g, 92% yield), mp: 148-153° C. ¹H NMR (400 MHz, DMSO-d₆) δ 10.10 (s, 1H), 9.06 (d, J=2.7 Hz, 1H), 8.94 (s, 1H), 8.55 (dd, J=4.7, 1.4 Hz, 1H), 8.22 (ddd, J=8.4, 2.8, 1.4 Hz, 1H), 7.55 (dd, J=8.4,4.7 Hz, 1H), 6.64 (dd, J=17.0, 10.2 Hz, 1H), 6.30 (dd, 17.1,2.0 Hz, 1H), 5.80 (dd, J=10.2, 2.0 Hz, 1H). ¹³C NMR (101 MHz, DMSO-d₆) δ 162.95, 147.56, 139.50, 135.46, 133.66, 130.39, 127.49, 125.56, 124.23, 122.56, 119.91. ESIMS: m/z 249.1 ([M+H]⁺).

### Example 6

### Preparation of N-(3-chloro-1-(pyridin-3-yl)-1H-pyrazol-4-yl)-3-((3,3,3-trifluoropropyl)thio)propanamide (Vc)

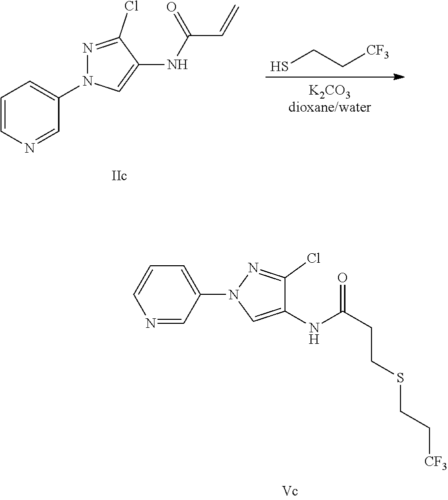 [SMILES: C=CC(=O)Nc1cn(-c2cccnc2)nc1Cl ; FC(F)(F)CCS ; O=C(CCSCCC(F)(F)F)Nc1cn(-c2cccnc2)nc1Cl]

To a round bottom flask was added K₂CO₃ (1.22 g, 8.83 mmol), water (4 mL), and dioxane (4 mL). 3,3,3-Trifluoropropane-1-thiol (0.70 g, 5.42 mmol, 90%) was added, and the mixture was stirred for 5 min. The above prepared mixture was added to a 50-mL round bottom flask containing N-(3-chloro-1-(pyridin-3-yl)-1H-pyrazol-4-yl)acrylamide (1.1 g, 4.42 mmol), dioxane (8 mL), and water (8 ml). The reaction mixture was stirred at 50° C. for 1 h, at which point HPLC analysis indicated that the reaction was complete. The solution was cooled to room temperature and poured into a separatory funnel containing EtOAc (50 mL) and saturated NaHCO₃ solution (10 mL). The organic layer was separated, and the aqueous phase was extracted with EtOAc (50 mL). The combined organics were washed with brine (25 mL), dried over anhydrous Na₂So₄ and concentrated under reduced pressure to afford a white solid, which was suspended in MTBE/hexane (25 mL, 1:9) and collected by filtration to afford 1.52 g of an off-white solid. The solid was suspended in MTBE/hexane (50 mL, 1:9) and stirred for 1 h. The solid was collected by filtration and rinsed with hexane (10 mL) to afford the desired product N-(3-chloro-1-(pyridin-3-yl)-1H-pyrazol-4-yl)-3-((3,3,3-trifluoropropyl)thio)-propanamide (Vc) as a white solid (1.27 g, 98% purity, 76% yield). ¹H NMR (400 MHz, DMSO-d₆): 9.92 (s, 1H), 9.05 (s, 1H), 8.86 (s, 1H), 8.53 (d, J=4.6 Hz, 1H), 8.21 (d, J=9.5 Hz, 1H), 7.54 (dd, J=8.2 Hz, 4.8 Hz, 1H), 2.85 (t, J=7.0 Hz, 2H), 2.73 (m, 4H), 2.58 (m, 2H). ¹³C NMR (101 MHz, DMSO-d₆): 169.3, 147.4, 139.4, 135.4, 133.3, 126.6 (q, J=296 Hz), 125.4, 124.2, 122.2, 120.0, 35.1, 33.4 (q, J=27.2 Hz), 26.7, 23.0 (q, J=3.3 Hz). ESIMS m/z 379.0 ([M+H]⁺).

### Example 7

### Preparation of N-(3-chloro-1-(pyridin-3-yl)-1H-pyrazol-4-yl)-N-ethylacrylamide (IId)

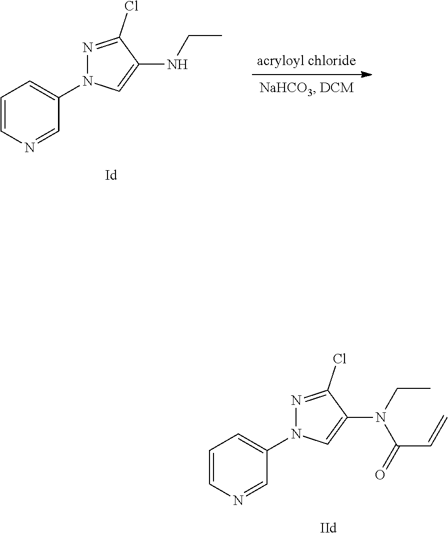 [SMILES: CCNc1cn(-c2cccnc2)nc1Cl ; C=CC(=O)N(CC)c1cn(-c2cccnc2)nc1Cl]

A 4-neck, 500-mL round bottom flask was charged with 3-chloro-N-ethyl-1-(pyridin-3-yl)-1H-pyrazol-4-amine (20.0 g, 90 mmol), and DCM (200 mL). NaHCO₃ (18.86 g, 225 mmol) was added, and the reaction was cooled to <5° C. Acryloyl chloride (8.76 mL, 108 mmol) was added dropwise at <10° C. The reaction was stirred at 20° C. for 2 h, at which point HPLC analysis indicated that the reaction was complete. The reaction was diluted with water (200 mL) (off-gassing) and the layers were separated. The aqueous layer was extracted with DCM (100 mL) and the combined organic layers were concentrated to dryness to afford a light brown oil, which was purified by column chromatography (330 g silica, 0-50% EtOAc/hexanes over 5 column volumes, hold at 50% for 5 column volumes). The fractions containing pure product were concentrated to dryness to afford N-(3-chloro-1-(pyridin-3-yl)-1H-pyrazol-4-yl)-N-ethylacrylamide (IM) as a white solid after drying under vacuum at 20° C. for 2 days (15.8 g, 64%). mp: 81-82° C. ¹H NMR (400 MHz, CDCl₃) δ 8.97 (d, J=2.7 Hz, 1H), 8.71-8.53 (m, 1H), 8.06 (ddd, J=8.3, 2.8,1.5 Hz, 1H), 7.98 (s, 1H), 7.46 (dd, J=8.3,4.7 Hz, 1H), 6.43 (dd, 16.7, 1.9 Hz, 1H), 6.18 (dd, J=16.8, 10.3 Hz, 1H), 5.75-5.50 (m, 1H), 3.78 (q, J =7.2 Hz, 2H), 1.20 (t, J=7.1 Hz, 3H). ¹³C NMR (101 MHz, CDCl₃) δ 165.77, 148.59, 141.12, 139.99, 135.65, 128.92, 127.58, 126.39, 126.22, 124.07, 123.79, 44.06, 13.02. ESIMS: m/z 277.1 ([M+H]⁺).

### Example 8

### Preparation of N-(3-chloro-1-(pyridin-3-yl)-1H-pyrazol-4-yl)-N-ethyl-3-((3,3,3-trifluoropropyl)thio)propanamide (Vd)

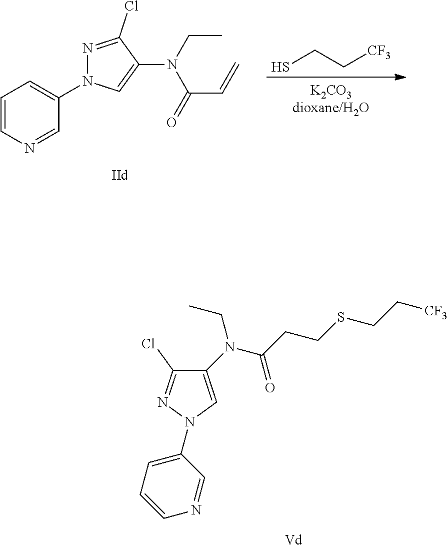 [SMILES: C=CC(=O)N(CC)c1cn(-c2cccnc2)nc1Cl ; FC(F)(F)CCS ; CCN(C(=O)CCSCCC(F)(F)F)c1cn(-c2cccnc2)nc1Cl ; "Vd"]

To a stirred solution of N-(3-chloro-1-(pyridin-3-yl)-1H-pyrazol-4-yl)-N-ethylacrylamide (0.4 g, 1.44 mmol) and K₂CO₃ (0.4 g, 2.89 mmol) in a mixture of dioxane (5 mL) and water (5 mL) was added 3,3,3-trifluoropropane-1-thiol (0.34 g, 2.6 mmol). The reaction mixture was stirred at room temperature for 2 h and monitored by HPLC. The reaction mixture was diluted with EtOAc (25 mL), the layers were separated and the aqueous layer was extracted with EtOAc (3×25 mL). The combined organic layers were washed with brine (50 mL), dried over anhydrous Na₂SO₄, filtered and concentrated to give 0.45 g of a reddish oil in 93% purity. The crude oil was purified by column chromatography (0-100% EtOAc/hexane) to afford the desired product as an off-white solid (0.40 g, 97.2% purity, 68% yield). ¹H NMR (400 MHz, CDCl₃): 8.94 (s, 1H), 8.61 (d, J=4.7 Hz, 1H), 8.04 (d, J=8.3 Hz, 1H), 7.97 (d, J=1.5 Hz, 1H), 7.45 (dd, J=8.3 Hz, 4.8 Hz, 1H), 3.70 (q, J=7.0 Hz, 2H), 2.82 (t, J=7.2 Hz, 2H), 2.69-2.59 (m, 2H), 2.43 (t, J=7.2 Hz, 2H), 2.40-2.27 (m, 2H), 1.15 (t, J=7.1 Hz, 3H). ESIMS m/z 406.9 ([M+H]⁺).

1. A process comprising
(a) contacting a compound of the formula I 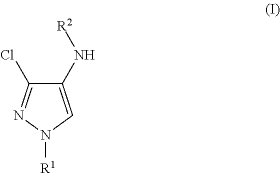
wherein R¹ is H or pyridin-3-yl; and R² is H or C₁-C₆ alkyl, with a compound of the formula X—C(O)CH═CH₂, wherein X is a leaving group, in the presence of a base and a solvent to provide a compound of the formula II 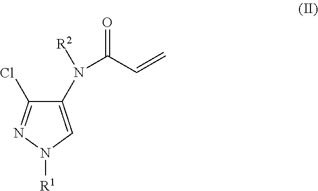
wherein R¹ is H or pyridin-3-yl; and R² is H or C₁-C₆ alkyl.

2. The process of claim 1, wherein R¹ is H.

3. The process of claim 1, wherein R¹ is pyridine-3-yl.

4. The process of claim 1, wherein R² is H.

5. The process of claim 1, wherein R² is ethyl.

6. The process of claim 1, wherein R³ is 3,3,3-trifluoropropyl.

7. The process of claim 1, wherein the base in step (a) is an inorganic base.

8. The process of claim 7, wherein the inorganic base in step (a) is selected from the group consisting of sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄) and potassium phosphate (K₃PO₄).

9. The process of claim 8, wherein the inorganic base in step (a) is NaHCO₃.

10. The process of claim 1, wherein the solvent in step (a) is methylene dichloride (DCM), N,N-dimethylformamide (DMF), tetrahydrofuran (THF), ethyl acetate (EtOAc), acetone, acetonitrile (CH₃CN), or dimethylsulfoxide (DMSO).

11. The process of claim 10, wherein the solvent in step (a) is EtOAc or DCM.

12. A process comprising
(b) contacting a compound of the formula II 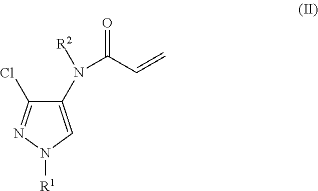
wherein R¹ is H or pyridin-3-yl; and R² is H or C₁-C₆ alkyl, with a compound of the formula HSR³, wherein R³ is C₁-C₆ alkyl optionally substituted with one or more halogen atoms or C₁-C₃ alkyl-C₃-C₆ cycloalkyl optionally substituted with one or more halogen atoms, in the presence of a base and a solvent to provide the compound of the formula V. 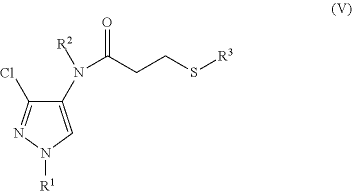
wherein R¹ is H or pyridin-3-yl; R² is H or C₁-C₆ alkyl; and R³ is C₁-C₆ alkyl optionally substituted with one or more halogen atoms or C₁-C₃ alkyl-C₃-C₆ cycloalkyl optionally substituted with one or more halogen atoms.

13. The process of claim 12, wherein the base in step (b) is selected from the group consisting of sodium bicarbonate (NaHCO₃), sodium carbonate (NaHCO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄) and potassium phosphate (K₃PO₄).

14. The process of claim 13, wherein the base in step (b) is K₂CO₃.

15. The process of claim 12, wherein the solvent in step (b) is a mixture of an organic solvent and water.

16. The process of claim 15, wherein the solvent in step (b) is a mixture of water and dioxane.

17. The process of claim 12, wherein the solvent in step (b) comprises acetone, acetonitrile, dioxane, DMSO, or THF.

18. A process for preparing a compound of the formula V 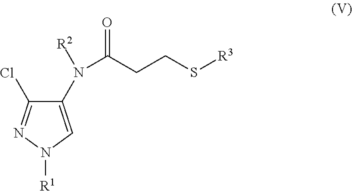
wherein R¹ is H or pyridin-3-yl; R² is H or C₁-C₆ alkyl; and R³ is C₁-C₆ alkyl optionally substituted with one or more halogen atoms or C₁-C₃ alkyl-C₃-C₆ cycloalkyl optionally substituted with one or more halogen atoms, comprising
(a) contacting a compound of the formula I 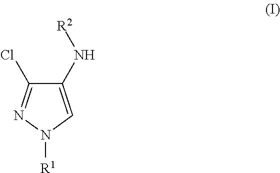
wherein R¹ is H or pyridin-3-yl; and R² is H or C₁-C₆ alkyl, with a compound of the formula X—C(O)CH═CH₂, wherein X is a leaving group, in the presence of a base and a solvent to provide a compound of the formula II 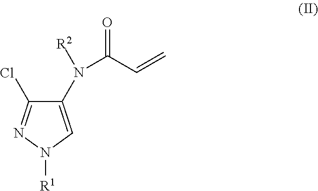
wherein R¹ is H or H or pyridin-3-yl; and R² is H or C₁-C₆ alkyl; and
(b) contacting a compound of the formula II 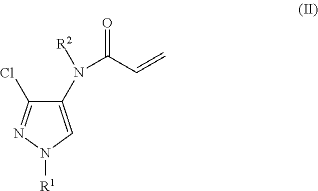
wherein R¹ is H or pyridin-3-yl; and R² is H or C₁-C₆ alkyl, with a compound of the formula HSR³, wherein R³ is substituted or unsubstituted C₁-C₆ alkyl or substituted or unsubstituted C₁-C₃ alkyl-C₃-C₆ cycloalkyl optionally substituted with one or more halogen atoms, in the presence of a base and a solvent to provide the compound of the formula V.

19. A compound of the formula

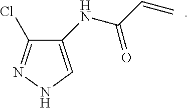

20. A compound of the formula

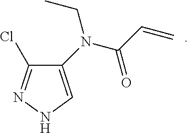

21. The process of claim 12, wherein the base in step (b) is an organic base.

22. The process of claim 21, wherein the base is selected from the group consisting of triethylamine (TEA), diisopropylethylamine (DIPEA), and pyridine.

23. The process of claim 12, wherein the base is used in excess compared to the compound of the formula II.

24. The process of claim 12, wherein the thiol reagent is a C₁-C₆ alkyl substituted with from 1 to 3 fluorine atoms.

25. The process of claim 24, wherein the thiol reagent is 3,3,3-trifluoropropane-1-thiol.

26. The process of claim 12, wherein the thiol reagent in used in excess compared to the compound of the formula II.

27. The process of claim 12, wherein the solvent in step (b) is a polar aprotic solvent or a water miscible solvent.

28. The process of claim 27, wherein the solvent comprises methylene dichloride (DCM), N,N-dimethylformamide (DMF), or ethyl acetate (EtOAc).

29. The process of claim 12, wherein the reaction is carried out at a temperature of between about 25° C. to about 75° C.

30. The process of claim 12 wherein R₁ is H, R₂ is H, R₃ is 3,3,3-trifluoropropyl, the base in step (b) is K₂CO₃, and the solvent in step (b) is a mixture of water and dioxane.

31. The process of claim 12, wherein the reaction is carried out at a temperature of 50° C. and a resulting solution is cooled to room temperature and poured into EtOAc and NaHCO₃.

32. The process of claim 31, wherein the resulting organic layer is separated, and the aqueous phase is extracted with EtOAc.

33. The process of claim 32, wherein the combined organics are washed with brine and dried over anhydrous Na₂SO₄ and concentrated under reduced pressure to afford a white solid.

34. The process of claim 33, wherein the white solid is suspended in MTBE and hexane, and a resulting solid is collected by filtration and rinsed with hexane to afford the compound of formula V.
